(19) United States
(12) Patent Application Publication
(10) Pub. No.: US 2026/0228480 A1
(43) Pub. Date: Aug. 6, 2026

(54) A METHOD FOR STORING AND TRANSPORTING FORENSIC EVIDENCE, AND CONTAINER COMPRISING FORENSIC EVIDENCE

(71) Applicant: Martin Jan Peter EVERSDIJK, Nieuw Vennep (NL)

(72) Inventor: Martin Jan Peter EVERSDIJK, Nieuw Vennep (NL)

(21) Appl. No.: 19/154,907

(22) PCT Filed: Feb. 5, 2024
(86) PCT No.: PCT/NL2024/050058

§ 371 Date: Aug. 8, 2025

(30) Foreign Application Priority Data
Feb. 8, 2023 (NL) 2034111

(51) Int. Cl. G06K 19/077; G01N 1/00; G01N 1/02

(52) U.S. Cl. CPC G06K 19/07798; G01N 1/02; G01N 2001/007

(57) ABSTRACT

A method for storage and transportation of forensic evidence in a container, wherein said container has a bottom wall, a side wall and an opening. The forensic evidence is collected at a forensic location and is inserted in the container, and a cap is attached to the container for sealing the opening of container, which is then transported to a remote location for storage of investigation. An NFC tag is used, in which a first part of the NFC tag is attached to the wall of the container, and a second part of the NFC tag is attached to the cap. A GPS device is used that includes a GPS unit for determining the location of the GPS device and an NFC communication unit for reading NFC tag identifier data from and/or store location data on the NFC tag.

### FIELD OF INVENTION

The present invention relates to a method for storing and transporting forensic evidence in a container, wherein said container has a bottom wall, a side wall and an opening, wherein the forensic evidence is collected at a forensic location and is inserted in the container, and wherein a cap is attached to the container for sealing the opening of said container, and wherein said sealed container is transported to a remote location for storage or investigation.

Such a method is disclosed in WO 2016/124229 A1, which is incorporated herein by reference.

### BACKGROUND

The known method is used in the case of criminal offences involving shooting of firearms for collecting the empty case of a bullet found at the site where the criminal offense has been committed. This is done by removing the cap from the sterile, DNA free and GSR (Gun Shot Residue) free container, picking up the empty case and subsequently placing it back in the container. The sealed container is then transported to a laboratory, where the empty case is examined for, for instance, propellant traces and indications of mechanical impact form the firearm from which it is fired.

Another such method is disclosed in WO 2016/023891 A1, which is incorporated herein by reference. In said method a certain critical amount of biological material such as saliva, sperm, blood or epidermis cells is safeguarded to ensure that a DNA profile can be established. These biological samples are collected by touching the surface where the biological sample is placed with for example a swab. After the collection of the biological sample, the swab is stored in the container to safeguard the sample and then transported to a laboratory to establish a DNA profile.

### SUMMARY

The object of the invention is to provide a method of the above kind which enhances the evidential value the stored evidence, which is easier to perform and/or which reduces the risk of mistakes being made.

To this end, a method according to the preamble is characterized in that an NFC tag is used, wherein after said cap is attached to the container for sealing the container a first part of said NFC tag is attached to said wall of the container, and a second part of said NFC tag is attached to the cap, and wherein a GPS device is used, said GPS device comprising a GPS unit for determining the location of the GPS device and an NFC communication unit for reading NFC tag identifier data from and/or store location data on said NFC tag, wherein said GPS device determines the location of the GPS device at the forensic location, and the GPS device communicates and stores the determined location on the NFC tag, or the GPS device reads the NFC tag identifier data from the NFC tag and stores the NFC tag identifier data together with the determined location in an electronic storage medium.

When shooting incidents occur, the position of the bullet cartridge can provide important information about the position of the shooter. This information can be crucial for the reconstruction and testimony of the shooting incident. The use of an NFC tag that seals the cap to the container can provide this information and can be used as a security. NFC tags are widely available, for instance in the form of stickers, and are each provided with an NFC chip. NFC chips comprise memory that is wirelessly readable (for reading stored information such as a unique identifier) and most NFC chips are also wirelessly writable (for storing additional information).

Preferably, the GPS device communicates and stores the determined location on the NFC tag.

In this manner the forensic location can be stored on the container itself at the forensic location, rather than in an external memory, and this data on the container can at any time thereafter be determined by using any NFC reader device, which does not need to be linked to data stored in the GPS device or an external database.

Preferably, said NFC tag is provided in the form of a self-adhesive sticker.

Preferably, said sticker forms a tamper evident seal, which is arranged to be visually deformed or damaged when it is removed after being attached to a surface.

According to a preferred embodiment, said forensic evidence is a biological sample, and wherein said cap is provided with a swab, such that said biological sample is held in said container when the cap is attached to the container.

According to an alternative preferred embodiment, said forensic evidence is an empty case of a firearm bullet cartridge for forensic investigation, wherein said case comprises a cylindrical shell which is substantially closed at one end, and wherein said cap is provided with holding means which are inserted in the empty case, which restrict lateral movement of the empty case in said container when the cap is attached to the container.

The present invention also relates to a container comprising forensic evidence collected at a forensic location, wherein said container has a bottom wall, a side wall and an opening, wherein a cap is attached to the container for sealing the opening of said container, wherein a first part of an NFC tag is attached to said wall of the container, and a second part of said NFC tag is attached to the cap.

Preferably, a GPS location of a forensic location is stored on the NFC tag.

Preferably, said NFC tag is provided in the form of a self-adhesive sticker.

Preferably, said sticker forms a tamper evident seal, which is arranged to be visually deformed or damaged when it is removed after being attached to a surface.

According to a preferred embodiment, said forensic evidence is a biological sample, wherein said cap is provided with a swab, such that said biological sample is held in said container when the cap is attached to the container.

According to an alternative preferred embodiment, said forensic evidence is an empty case of a firearm bullet cartridge for forensic investigation, wherein said case comprises a cylindrical shell which is substantially closed at one end, and wherein said cap is provided with holding means arranged to be inserted in the empty case for restricting lateral movement of the empty case in said container. Preferably, said holding means comprise a pin.

In that manner the empty case is restricted in lateral movement, whereby it cannot touch the wall of the container, provided the diameter of the container is large enough.

Preferably, said holding means comprise clamping means for clamping the closed end of said empty case against the bottom wall of the container.

In that manner the closed end of said empty case can be clamped against the bottom wall of the container when the cap is attached to the container, such that it cannot move and whereby the wall of the case cannot touch the wall of the container.

Preferably, said clamping means comprise a pin which is arranged to be inserted into the empty case and which pin is arranged to be pushed against said bottom wall. Preferably, the outer end part of the pin has a roughened surface. The outer end is roughened to deal with GSR (Gun Shot Residue). Preferably, said pin extends from the centre of said cap. Preferably, said pin is provided with a resilient part. Preferably, said resilient part comprises a spring. Preferably, said resilient part is compressible by at least 3 mm, preferably 5 mm. Preferably, said resilient part or spring extends from the cap and the pin extends from the resilient part or spring.

Preferably, the container is a tubular container.

Preferably, the container is made of a transparent material.

Thereby, the case can be photographed while in the container.

Preferably, the container has a length of 3-5 cm, preferably 3.5-4.5 cm.

Preferably, the container has a diameter of 2-3 cm, preferably 2.3-2.8 cm.

Preferably, said pin has a diameter of 0.3-0.4 cm, preferably 0.33-0.37 cm.

Preferably, the distance between the outer side surface of the pin and the inner surface of the side wall of the container extending on the opposite side of the pin is 0.8-1.7 cm, preferably 1.0-1.5 cm, more preferably 1.1-1.3 cm. By choosing said dimensions the device is suitable for empty cases of the most regular firearm bullet cartridges (for instance .45 caliber firearm bullet cartridges, of which the inner case diameter is 1.16 cm), such that they cannot touch the inner surface of the side wall of the container. Larger dimensions for larger calibers are of course possible.

Preferably, said bottom wall of said container is provided with ribs for maintaining a distance between the surface of said bottom wall and the closed end of the empty case.

Finally, the present invention relates to an NFC tag in the form of a self-adhesive sticker, wherein said sticker is arranged to form a tamper evident seal, which is arranged to be visually deformed or damaged when it is removed after being attached to a surface.

Such a tamper evident NFC sticker is in particular useful for use in the above described method.

### BRIEF DESCRIPTION OF THE DRAWINGS

The present invention will now be illustrated with reference to the drawing where FIG. 1 shows a perspective view of the parts that constitute a device for storage and transportation of an empty case of a firearm bullet cartridge for forensic investigation;

FIG. 2 shows a perspective view of an assembled device comprising the parts of FIG. 1; and

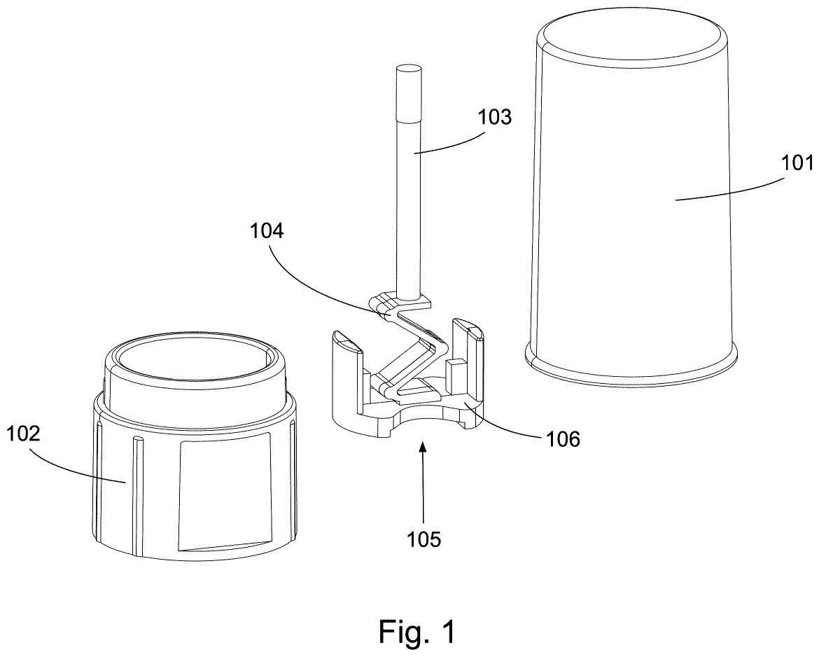

FIGS. 3A and 3B show perspective views of the device of FIG. 2 with an NFC tag seal.

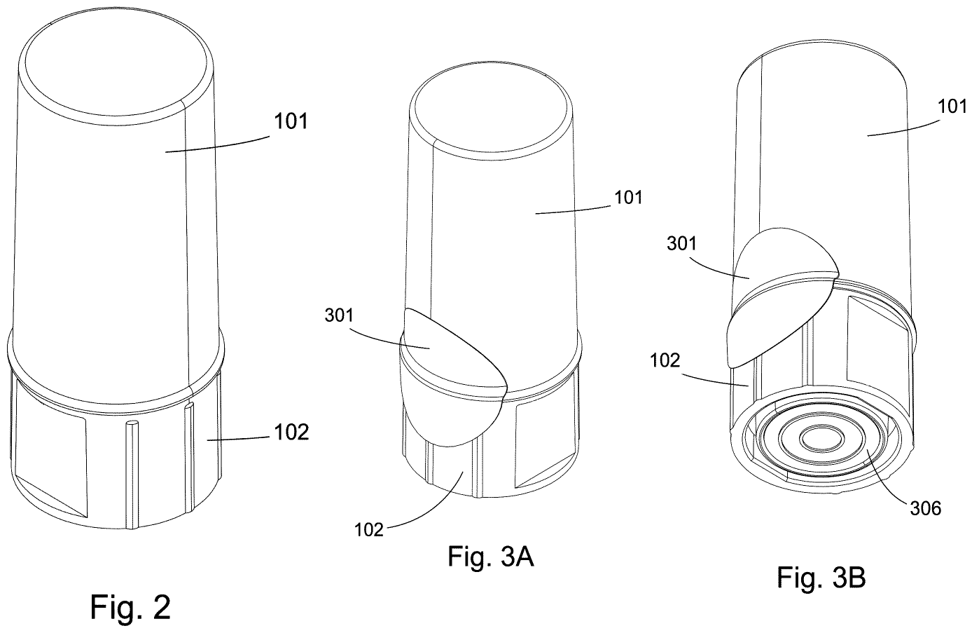

### DETAILED DESCRIPTION

With reference to FIG. 1 a device for storage and transportation of an empty case of a firearm bullet cartridge for forensic investigation comprises a tubular container 101, which is made for instance of a transparent plastic material, and a cap 102, which is in principle capable of sealing the container 101 at one end. A base member 106 is attached to the inner side of the cap 102, and a pin 103 is attached to the base member 102 by means of a compressible spring 104. The cap 102 is provided with clamping portion having a radial outer surface that is dimensioned to tightly fit in the container 101 such that it is clamped by the radial inner surface thereof.

Alternative closure methods, such as by screw or snap means, are of course possible. In the closed condition the pin 103 with the empty case push against the inner side of the bottom wall of the container 101. In the closed condition the pin 103 with the empty case may push against ribs provided on the bottom wall of the container 101, as disclosed in WO 2016/124229 A1. In that embodiment the ribs may extend in radial direction from the centre of the bottom wall. Thereby the empty shell cannot move when being transported.

The outer end part of the pin 103 has a roughened surface, in order to collect GSR (Gun Shot Residue) from the empty case.

A recess 105 is provided in the base member 106 for accomodating dehumidifier means, such as a capsule (not shown) with a sorbent material such as silica gel granules. Thereby the empty case is dried and decay of evidence is prevented.

In an alternative embodiment the container may be provided with a ventilating opening, for instance in the outer end wall of the cap 102. The ventilating opening may be covered by a filter, which allows moist to exit the container, but block anything else to pass, in particular skin cells. The end wall of cap 102 in that case is partially open to allow air and moist to pass through the opening and filter.

The outer side of the end wall of cap 102 may be provided with ribs, as disclosed in WO 2016/124229 A1, in order to allow air and moist to pass through to the environment when the cap is placed on a surface.

The most relevant dimensions of this preferred embodiment are as follows. The inner length of the container 101 is 5 cm, the inner diameter of the container 101 is 2.6 cm, the length of the pin 3 (measured from the outer end of the spring 104) is 3.5 cm, and the diameter of the pin 103 is 0.35 cm. This preferred embodiment is therefore especially suited for up to .45 caliber firearm bullet cartridges, of which the case diameter is 1.1 cm. Larger dimensions for larger calibers are of course possible.

In use, for example at the site where a criminal offense involving a shooting has been committed, the cap 102 with pin 103 is removed from the container, and the pin 103 is inserted in the opening of the empty shell such that it can be lifted from the ground without touching the shell. Subsequently the cap 2 with the empty case on the pin 103 is inserted into the container 101 and the cap 102 is fixed to the container, whereby the empty case is immobilized by clamping the end wall thereof, against the ribs that may be provided thereon. Any moisture contained in or on the empty case can subsequently evaporate and be absorbed by the silica gel capsule in the recess 105. In an alternative embodiment the moisture can exit the container via a filter in a ventilating opening which may be for instance provided in the bottom 306 of the cap 102.

After the cap 102 is screwed onto the container 101 an adhesive NFC tag 301 in the form of a sticker is placed on the container 101 and the cap 102, such that a first part of the NFC tag sticks to the container 101 and a second part of the NFC tag sticks to the cap 102. Thereby a tamper evident closure may be obtained. Preferably the sticker is a tamper evident sticker that deforms or is damaged when trying to pull it off the container.

A GPS device comprising a GPS unit is then used for determining the location of the GPS device at the forensic location. An NFC communication unit in the GPS device stores the location data on said NFC tag. The GPS location data on the container can at any time thereafter be determined by using any NFC reader device.

Alternatively, the GPS device reads the NFC tag identifier data from the NFC tag and stores the NFC tag identifier data together with the determined location in an electronic storage medium in the GPS device or wirelessly at a remote location.

The GPS device may be a mobile device such as a smart phone or a tablet, with software that is arranged to determine the GPS position and to write the determined GPS position to the NFC chip.

The invention has thus been described by means of preferred embodiments. It is to be understood, however, that this disclosure is merely illustrative. Various details of the structure and function were presented, but changes made therein, to the full extent extended by the general meaning of the terms in which the appended claims are expressed, are understood to be within the principle of the present invention. The description and drawings shall be used to interpret the claims. The claims should not be interpreted as meaning that the extent of the protection sought is to be understood as that defined by the strict, literal meaning of the wording used in the claims, the description and drawings being employed only for the purpose of resolving an ambiguity found in the claims. For the purpose of determining the extent of protection sought by the claims, due account shall be taken of any element which is equivalent to an element specified therein. An element is to be considered equivalent to an element specified in the claims at least if said element performs substantially the same function in substantially the same way to yield substantially the same result as the element specified in the claims.

1. A method for storing and transporting forensic evidence in a container, comprising:
providing a container having a bottom wall, a side wall and an opening,
collecting the forensic evidence at a forensic location and is inserted in the container, wherein a cap is attached to the container for sealing the opening of said container,
transporting said sealed container to a remote location for storage or investigation,
using an NFC (near field communication) tag,
after attaching said cap to the container for sealing the container, attaching a first part of said NFC tag to said wall of the container, and attaching a second part of said NFC tag to the cap, and
using a GPS device, said GPS device comprising a GPS unit for determining the location of the GPS device and an NFC communication unit for reading NFC tag identifier data from and/or store location data on said NFC tag,
determining, using said GPS device, the location of the GPS device at the forensic location, and
communicating and storing, with the GPS device, the determined location on the NFC tag, or
reading, with the GPS device the NFC tag identifier data from the NFC tag and storing the NFC tag identifier data together with the determined location in an electronic storage medium.

2. The method of claim 1, wherein the GPS device communicates and stores the determined location on the NFC tag.

3. The method of claim 2, wherein said NFC tag is provided in the form of a self-adhesive sticker.

4. The method of claim 3, wherein said sticker forms a tamper evident seal, which is arranged to be visually deformed or damaged when it is removed after being attached to a surface.

5. The method of claim 1, wherein said forensic evidence is a biological sample, and wherein said cap is provided with a swab, such that said biological sample is held in said container when the cap is attached to the container.

6. The method of claim 1, wherein said forensic evidence is an empty case of a firearm bullet cartridge for forensic investigation, wherein said case comprises a cylindrical shell which is substantially closed at one end, and wherein said cap is provided with holding means which are inserted in the empty case, which restrict lateral movement of the empty case in said container when the cap is attached to the container.

7. A container comprising forensic evidence collected at a forensic location, comprising:
a container has a bottom wall, a side wall and an opening,
attaching a cap to the container for sealing the opening of said container, attaching a first part of the NFC tag to said wall of the container, and attaching a second part of said NFC tag to the cap.

8. The container of claim 7, wherein a GPS location of a forensic location is stored on the NFC tag.

9. The container of claim 8, wherein said NFC tag is provided in the form of a self-adhesive sticker.

10. The container of claim 9, wherein said sticker forms a tamper evident seal, which is arranged to be visually deformed or damaged when it is removed after being attached to a surface.

11. The container of claim 7, wherein said forensic evidence is a biological sample, wherein said cap is provided with a swab, such that said biological sample is held in said container when the cap is attached to the container.

12. The container of claim 7, wherein said forensic evidence is an empty case of a firearm bullet cartridge for forensic investigation, wherein said case comprises a cylindrical shell which is substantially closed at one end, and wherein said cap is provided with holding means arranged to be inserted in the empty case for restricting lateral movement of the empty case in said container.

13. The container of claim 12, wherein said holding means comprise a pin.

14. The container of claim 7, wherein the container is a tubular container.

15. An NFC tag in the form of a self-adhesive sticker, wherein said sticker is arranged to form a tamper evident seal, which is arranged to be visually deformed or damaged when it is removed after being attached to a surface.
